(19) United States
(12) Patent Application Publication
(10) Pub. No.: US 2013/0059391 A1
(43) Pub. Date: Mar. 7, 2013

(54) TEST PIECE FOR HEAVY METAL ION, PROCESS FOR DETECTING HEAVY METAL ION, KIT AND SENSOR

(71) Applicants: Xiaoke Zhang, Shanghai (CN); Yongming Li, Shanghai (CN); Hisashi Kajiura, Shanghai (CN)

(72) Inventors: Xiaoke Zhang, Shanghai (CN); Yongming Li, Shanghai (CN); Hisashi Kajiura, Shanghai (CN)

(73) Assignee: SONY CORPORATION, Tokyo (JP)

(21) Appl. No.: 13/594,799

(22) Filed: Aug. 25, 2012

(30) Foreign Application Priority Data
Sep. 2, 2011 (CN) 201110258203.7

(51) Int. Cl. G01N 31/22; G01N 21/75; G01N 33/20; G01N 30/96

(52) U.S. Cl. 436/73; 422/69; 422/430; 422/424

(57) ABSTRACT

The invention provides a test piece for detecting heavy metal ions in an aqueous system to be detected, comprising a substrate, a polymer coating layer and a layer of heavy metal ion-detecting agent, wherein the polymer coating layer is provided such that the surface of the test piece is hydrophobic. The invention further provides a process for detecting heavy metal ions in an aqueous system, a kit comprising the heavy metal ion test piece and a sensor. A portable test piece and/or a device can be provided by the test piece according to the invention, so as to detect the heavy metal ions in a convenient, efficient and rapid manner.

### TECHNICAL FIELD

This invention relates to a test piece for detecting heavy metal ions in an aqueous liquid, a process of detecting heavy metal ions using the test piece, a portable kit for detecting heavy metal ions by the detection process, and the detect sensor for identifying heavy metal ions.

### BACKGROUND

The heavy metal (ion) pollution refers to the environmental pollution caused by heavy metals or their compounds. The increase of the heavy metal content in the environment, especially in the case of heavy metal pollution in an aqueous system, is mainly due to human factors, such as mining, waste gas emission, sewage irrigation and the use of heavy metal-contaning products, which results in the deterioration of environmental quality.

A commonly used method for the detection of heavy metal ions in an aqueous system is to add a heavy metal ion detecting agent to a sample to be detected, wherein the metal ion detecting agent binds the heavy metal ion to form a colored complex, that is, the existence of the heavy metal ion can be determined from the color change of the system. Because of the specificity of the color of the heavy metal ion detecting agent-heavy metal ion complex, the species and concentration of the heavy metal ion can be determined For example, by selecting dithizone as the heavy metal ion detecting agent, chelates can be formed by dithizone and heavy metal ions such as Hg(II), Pb(II), Cd(II) and Zn(II) existing in the assay sample. Since different metals form metal-dithizone complexes exhibiting different colors, low level of Hg(II), Pb(II), Cd(II), Zn(II) and the like in the aqueous solution can be detected by UV-Vis spectrometer (such as the Chinese National Standard Methods for Hg(II) (GB7469-87), Pb(II) (GB7470-87), Cd(II) (GB7471-87) and Zn(II) (GB7472-87)). However, because this method generally requires lab-scale instruments such as UV-Vis spectrometer and the identification of the detection results of the spectrometer requires expert experiences, it is not suitable for in situ rapid monitoring.

Moreover, the heavy metal ion detecting agents may be fixed in a substrate (also known as carrier) such as a test paper or a membrane for the detection of the heavy metal ions. When such a detection method is conducted by directly coating the detecting agent on the carrier such as a test paper or a membrane, the heavy metal ion detecting agent tends to leak (escape) due to the weak bonding between the heavy metal ion detecting agent and the carrier, thereby affecting the detection of the heavy metal ion. In addition, the heavy metal detecting agent on the test paper develops color during detection by reacting with the heavy metal ions in the aqueous solution to form a colored complex, so that the type of the heavy metal ion contained in the aqueous system can be determined by comparison with the Color Chart and meanwhile the concentration of the heavy metal ion can be qualitatively or quantitatively determined from the shade of the color. However, the heavy metal detecting agent such as dithizone, which is fixed on the substrate, is oil soluble and water insoluble, and thus when the test paper is immersed into the aqueous solution to be detected, the chelation between the heavy metal ions and these agents is inhibited due to their difference in solubility, which directly results in a typically higher detection limit of the test paper than that of the Chinese National Standard method.

Therefore, currently there is still a need for a heavy metal ion test piece which can be used to detect the small amount, even trace amount of heavy metal ions in an aqueous system in a simple, low cost, highly sensitive, highly reliable and stable manner. Meanwhile, it is required that the test piece is available for in situ detection, and is capable of detecting heavy metal ions with high sensitivity. Moreover, it is desired that the heavy metal ions can be not only qualitatively detected, but also quantitatively or semi-quantitatively detected.

### SUMMARY

The present invention provides a test piece for detecting heavy metal ions in an aqueous system, comprising a substrate, a polymer coating layer and a layer of heavy metal ion detecting agent, wherein the polymer coating layer is a coating layer of a hydrophobic or water-repellent polymer.

According to a particular embodiment, the invention provides a test piece for detecting heavy metal ions in an aqueous system, comprising a substrate, a polymer coating layer and a layer of heavy metal detecting agent, wherein the polymer in the polymer coating layer comprises, but not limited to, polyethylene; polyvinyl chloride; polystyrene; polypropylene; polybutene; polyisobutylene; polyformaldehyde; polyamides; polycarbonates; polylactic acid; polytetrafluoroethylene; poly(ethylene terephthalate); epoxy resins; phenolic resins; polyurethanes; polyacrylonitrile-butadiene-styrene; and poly(methyl methacrylate).

According to another particular embodiment, the invention provides a test piece for detecting heavy metal ions in an aqueous system, comprising a substrate, a polymer coating layer and a layer of a heavy metal ion-detecting agent, wherein the polymer is selected from polystyrene and poly(methyl methacrylate).

The present invention further provides a process for detecting heavy metal ions in an aqueous system: bringing the test piece for heavy metal ions into contact with the aqueous system to be detected; shaking the aqueous system to be detected so as to contact with the test piece sufficiently; and observing whether the color of the test piece is changed. Herein, an organic solvent capable of dissolving the heavy metal ion-detecting agent is contained in the aqueous system.

According to a particular embodiment, a process for detecting the heavy metal ions in an aqueous system is provided, wherein the process comprises adding an organic solvent capable of dissolving heavy metal ion-detecting agent into the aqueous system to be detected; bringing a test trip coated with a polymer coating and a heavy metal-detecting agent into contact with the aqueous system to be detected; shaking the aqueous system to be detected to contact with the test piece sufficiently; and observing whether the color of the test piece is changed.

The invention further provides a kit (test suite) for the detection of heavy metal ions in an aqueous system, comprising a test trip for the detection of the heavy metal ions in an aqueous system (solution), an organic solvent and a color chart.

According to a particular embodiment of the invention, a kit for the detection of heavy metal ions is provided, which comprises a test trip for the detection of the heavy metal ions in an aqueous solution, an organic solvent capable of dissolving the heavy metal ion-detecting agent, a sampling container, a color chart, wherein the color chart is the color chart corresponding to various heavy metal ion-detecting agents.

According to the invention, a test sensor for the detection for heavy metal ions is provided, which comprises a test trip for the detection of the heavy metal ions in an aqueous solution, a sample cell containing an organic solvent capable of dissolving the heavy metal ion-detecting agent, a color chart, and a test piece color identification sensor.

According to one embodiment of the invention, the provided test sensor for the detection of heavy metal ions comprises a test trip for the detection of the heavy metal ions in an aqueous solution, a sample cell containing an organic solvent capable of dissolving the heavy metal ion-detecting agent, a color chart and a test piece color identification sensor, wherein the test piece is coated with an heavy metal ion-detecting agent and a polymer coating.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will become more fully understood from the detailed description given hereinafter and the accompanying drawings which are given by way of illustration only, and thus are not limitative of the present invention and wherein:

FIG. 1 is a schematic graph for the detection principle of the heavy metal ions in an aqueous solution by using a dithizone test piece (test paper) coated with a lipophilic polymer coating.

FIG. 2 shows SEM micrographs of (a, b) a normal filter paper and (c, d) a filter paper having a polystyrene coating.

FIG. 3 shows selective adsorption ability of the filter paper having the polystyrene coating. In the Figure, the aqueous solution on the left side is the aqueous solution of the water soluble ink (water phase, red); the organic solution on the right side is the dithizone solution in carbon tetrachloride (oil phase, green). FIGS. 3a and 3b show photos of water droplets (left side, red) and oil droplets (right side, green) on (a) the normal paper and the filter paper having the polystyrene coating (the filter paper with a lipophilic coating) and (b) the filter paper having the polystyrene coating after removing the water droplet. FIGS. 3c, d are photos of the normal paper and the filter paper having the polystyrene coating after immersed into a layered oil-water mixing system (shown in FIG. 3c, the upper layer is an aqueous solution, the lower layer is an organic solution) and an emulsified oil-water mixing system (shown in FIG. 3d) and being taken out.

FIG. 4 shows capability of preserving dithizone for the filter paper substrates having different polymer coatings. FIG. 4 shows capability of preserving the heavy metal ion-detecting agent for the dithizone pieces made with polyvinylidene difluoride (PVDF), blank filter paper (0 wt %, PS), 2 wt % polystyrene solution and 8 wt % polystyrene solution after standing for 1 minute to 24 hours at room temperature under natural light in the air environment.

FIG. 5 shows the detection principle of Pb(II) in aqueous solution by using the dithizone test piece made by the filter paper substrate having the polymer coating. FIG. 5a shows the photo of the water droplets containing Pb(II) (water droplets on the left side of FIG. 5a) and the water/C₂Cl₄ mixing droplets containing Pb(II) (droplets on the right side of FIG. 5a) after standing on the surface of the dithizone piece for 2 min. FIG. 5b shows the photo of the dithizone piece after placing into an aqueous solution containing Pb(II) and shaking for 2 min. FIG. 5c shows the photo of the dithizone piece after placing into a water/C₂Cl₄ mixing solution containing Pb(II) (C₂Cl₄/water volume ratio=1/20) and shaking for 2 min. In FIG. 5c, dark (red) C₂Cl₄ droplets of Pb-dithizone complex adsorbed onto the surface of the test piece can be observed.

FIG. 6 shows the color changes of detecting different concentrations of Pb(II) ions in an aqueous solution by using the dithizone detecting agent piece without a polymer coating (Test piece 1), the dithizone test piece without a polymer coating (Test piece 2, the same to the Test piece 1) and the test piece having the polymer coating and the dithizone heavy metal ion detecting agent (Test piece 3). FIG. 6 (upper) shows photos of the color changes of test papers obtained after placing the Test piece 1 into aqueous Pb(II) solutions at different concentrations and reacting. FIG. 6 (middle) shows photos of the color changes of test papers obtained after placing the Test piece 2 into Pb(II) water/C₂Cl₄ mixing solutions at different concentrations and reacting. FIG. 6 (lower) shows photos of the color changes of test papers obtained after placing the Test piece 3 into Pb(II) water/C₂Cl₄ mixing solutions at different concentrations and reacting.

FIG. 7 shows detection of Cd(II) in water by using the dithizone test piece having the poymer coating.

FIG. 8 shows detection of Zn(II) in water by using the dithizone test piece made by the filter paper having the poymer coating.

FIG. 9 shows detection of Cu(II) in water by using the dithizone test piece made by the filter paper having the poymer coating.

FIG. 10 shows detection of Hg(II) in water by using the dithizone test piece made by the filter paper having the poymer coating.

FIG. 11 shows detection of the color change of high concentration of Pb(II) in the aqueous system by using the dithizone piece made by the filter paper having the polymer coating.

FIG. 12 shows selective adsorption ability for oil phase and ability of detecting the heavy metal ions of the test piece having the PMMA polymer coating. In FIG. 12, the left is the droplet of the (red) aqueous solution (water phase), and the right (green) is the droplet of the organic solution (oil phase). FIG. 12a shows the hydrophobicity (on the left side, droplet is present) and the hydrophilicity (on the right side, the droplet is absorbed) of the hydrophobic polymer coating; FIG. 12.b shows photos after removing the water droplets on the test piece having the PMMA coating (paper having the polymer coating); FIG. 12c shows photos for the dithizone test piece having the PMMA solution coating after dripping the deionized water/C₂Cl₄ or Zn(II)-containing aqueous solution/C₂Cl₄ mixture (with the volume ratio being 20/1) onto it, wherein water does not change its color on the dithizone test piece having PMMA coating while the solution containing Zn ion caused the color change of the test piece.

FIG. 13 shows a multi-sample detection in aqueous solutions of different heavy metal ions on the same dithizone test piece having the polymer coating. For FIG. 13a, the same test piece exhibits different colors for different types of heavy metal ions, and for FIG. 13b, the same test piece exhibits different colors for aqueous Pb(II) solutions at different concentrations.

FIG. 14 shows the dithizone pieces (test papers) made by normal A4 printing paper. FIGS. 14a and 14b show photos of water droplets and oil droplets on (a) the normal A4 paper (normal paper) and the A4 paper having a polymer coating, and (b) test paper after removing the water droplet. FIG. 14c shows multi-detection of water samples with different types of metal ions by using the dithizone test piece (test paper) having the polymer coating made by A4 paper, wherein different colors were shown, pink for Zn(II); amber for Cu(II); off-orange red for Pb(II); and light yellow for Cd(II).

FIG. 15 shows detection of Ag (I) using the rose red silver test piece having the polymer coating (test paper of polymer coating—rose red silver test reagent). (a) the Ag(I) test paper was made from a rose red silver solution and an A4 paper coated with the polymer coating (upper) or a normal A4 paper (lower). (b) Detection of Ag(I) was conducted by using the rose red silver test paper made by the filter paper having the polymer coating (test paper of polymer coating—rose red silver test reagent, upper) and using the rose red silver test paper made by the normal A4 paper (lower).

FIG. 16 shows stability test of the dithizone test piece prepared from the filter paper substrate having the polymer coating.

FIG. 17 shows a schematic graph for the principle of the heavy metal ion-detecting sensor.

FIG. 18 shows the change trend of R value (Pb(R)) and G value (Pb(G)) of the color of the dithizone test piece (test paper) read by the test piece color identification sensor after the detection of different concentrations of Pb(II), wherein Pb(R-G) is the difference of R value and G value.

### DETAILED DESCRIPTION

A Test Piece for the Detection of Heavy Metal Ions in an Aqueous Liquid.

A test piece for the detection of heavy metal ions in an aqueous liquid, comprising a substrate, a polymer coating layer and a layer of heavy metal ion-detecting agent, wherein the polymer coating layer is provided such that the surface of the test piece is hydrophobic.

The polymers which can endow the surface of the test piece with hydrophobicity comprise, but not limited to, at least one of polyethylene; polyvinyl chloride; polystyrene; polypropylene; polybutene; polyisobutylene; polyformaldehyde; polyamides; polycarbonates; polylactic acid; polytetrafluoroethylene; poly(ethylene terephthalate); epoxy resins; phenolic resins; polyurethanes; polyacrylonitrile-butadiene-styrene; and poly(methyl methacrylate). In the test piece according to the invention, the use of polystryrene and/or poly(methyl methacrylate) is preferable.

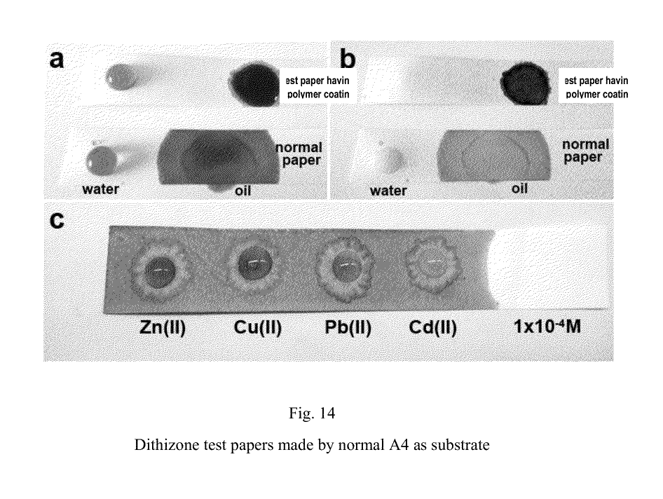

The substrate for forming the test piece can be any substrate material that can carry the heavy metal ion detecting agent, including glass sheet, film, adsorbing material such as cellulosic materials and the like, as long as it can carry the heavy metal ion detecting agent. Preferably, the substrate material can be any porous material with adsorbency, for example, a cellulosic material such as paper, including filter paper and normal printing paper (for example, A4 paper). There is no particular limitation to the shape of the substrate material, but a sheet, which has a high specific surface area, is preferred. Moreover, a sheet shaped substrate is preferred in view of portability and convenience of use. In practical use, the substrate can be designed to have suitable size and shape as required. Since the test piece having a polymer coating layer according to the invention has high sensitivity of detection, only very small volume is needed to effectively detect the heavy metal ions in an aqueous system (for example, as shown in FIG. 14). Usually, the substrate is preferably a strip or a tape due to the stability of preservation and sensitivity of detection of the test piece having a polymer coating layer according to the invention, and considering portability and convenience of use. The test piece according to the invention is small and thus portable and convenient in use.

The method for coating or applying the polymer coating on the substrate can be any means to apply a polymer on the substrate, such as the means of dip coating, spin coating, and the like. Preferably, the polymer to be coated is dissolved in its (good) solvent, especially an organic solvent, to form a polymer solution, such as a 1 wt % to 20 wt % polymer solution. The polymer solution is thereby applied to the substrate and dried to obtain the test piece having the polymer coating. There is no particular restraint on the thickness of the polymer coating, as long as the substrate is completely coated so that the coated test piece becomes hydrophobic.

The heavy metal detecting agent used for the test piece can be various heavy metal ion detecting agents commonly used. Preferably, the heavy metal ion detecting agent used for the invention is an organic heavy metal reagent (or called as an oil soluble organic heavy metal ion detecting agent), preferably an oil soluble organic heavy metal ion detecting agent, for example, dithizone, which forms a colored substance with a particular color based on the chelation between dithizone and heavy metal ions. These heavy metal ion detecting agents comprise, but not limited to, dithizone, 5-(4-Dimethylaminobenzylidene)rhodanine, diphenylcarbohydrazide, developers based on triphenylmethane (such as Victoria blue B, crystal violet, malachite green and the like).

These heavy metal ion detecting agents form complexes with heavy metal ions that specifically develop color. This can be used to identify the heavy metal ions in an aqueous system, including the heavy metal ions such as Hg(II), Pb(II), Cd(II), Zn(II), Cu(II) and the like that exist in the environment, especially in aqueous systems. Based on the color that is developed, the existence, type as well as the general level/concentration of the heavy metal ions in the system can be qualitatively and/or (semi-)quantitatively determined

The heavy metal ion detecting agents are specific to the color of a complex (chelate) of heavy metal ion detecting agent-heavy metal ion formed by a particular heavy metal ion. Based on the color appeared and the darkness of the color, the level of heavy metal ion pollution of the environment, that is, the concentration of the heavy metal ion in the system, can be determined A person of ordinary skill in the art is capable of identifying the heavy metal ion according to the specific color. For example, dithizone reacts with different heavy metal ions such as Hg(II), Pb(II), Cd(II), Zn(II), Cu(II) and the like to form complexes with different color, for example, the dithizone detecting agent develops yellow for Cd(II) and develops red for Pb(II). The heavy metal ion detecting agent (layer) on the test piece is preferably located on (within) the polymer coating on the test piece. The heavy metal ion detecting agent can be applied to the heavy metal ion detecting agent layer in any manner. The test piece having the polymer coating and the heavy metal ion detecting agent, which has a hydrophobic surface, can be obtained by coating the heavy metal ion detecting agent on (within) the polymer coating by applying the heavy metal ion detecting agent layer in situ on the test piece already coated with the polymer layer by applying the solution of the heavy metal ion detecting agent before the detection, and using after dried.

In the aqueous system to be detected, if there exist more than one heavy metal ion, pH value can be adjusted or a masking agent can be added to exclude other ions (interfering ions) other than the heavy metal ion to be detected as required in order to avoid interference. The masking agent can be a commonly used masking agent in the art, such as hydroxylamine hydrochloride, ethylenediamine tetraacetic acid (EDTA), potassium sodium tartrate, ammonium citrate, and the like, which can be chosen according to the prior art. When the target to be detected is a solid, for example, when it is desired to detect whether soil contains heavy metal ions, the target to be detected is pre-treated to be dissolved in water or a solvent system of water and an organic solvent that can dissolve the heavy metal ion detecting agent, so that the heavy metal ion in the target to be detected is dissolved in this aqueous system, and the test piece of the invention is used for detection.

Because the test piece of the application has the polymer coating and the organic heavy metal ion detecting agent, it has a hydrophobic surface. Preferably, the aqueous system to be detected contains an organic solvent, especially an organic solvent that can dissolve the heavy metal ion detecting agent and/or the heavy metal ion detecting agent-heavy metal ion chelate. Preferably, said organic solvent is immiscible with water. The amount of the organic solvent in the aqueous system is not particularly constrained as long as it is trace amount. Preferably the ratio of the organic solvent : aqueous solvent (the aqueous system) is 1:5 to 1:50 (volume ratio), preferably 1:10 to 1:50 (volume ratio). When the system does not contain any organic solvent, it is preferred that the organic solvent is added. Depending on the heavy metal ion detecting agent, alkane, for example C1-C6 alkane, benzene, toluene, chlorine-containing C1-C6 alkane, chlorine-containing C2-C6 alkene such as CCl₄, CHCl₃, and C₂Cl₄, can be used. C₂Cl₄ is preferred as required.

Because the test piece of the invention contains a hydrophobic polymer coating (that is, the surface of the test piece is hydrophobic), the binding strength between the organic heavy metal ion detecting agent and the test piece is elevated. Meanwhile, the polymer layer shields the air and the ultraviolet light in sunlight to some extent, enabling the heavy metal ion detecting agent in the test piece to be maintained for a longer period.

Due to the structure of the test piece described herein and the appeared hydrophobicity of its surface, without bound by any theory, a double extraction process occurs during the detection of the heavy metal ions with this test piece. Firstly, the polymer coating in the test piece favors selective adsorption of liquid with low surface free energy (oil phase) in liquid with high surface free energy (aqueous phase). Therefore, the test piece is modified by the polymer coating (to become lipophilic/hydrophobic). Said test piece leads to the first extraction in the aqueous system, wherein the organic heavy metal ion detecting agent in the surface of the test piece is dissolved in the organic solvent in the aqueous system (organic solvent/water=1/20 by volume ratio), which extracts the heavy metal ions in the aqueous solution to form heavy metal ion detecting agent-heavy metal ion complexes (chelates) which is soluble in the organic solvent, and said complexes have specific color (appearing to develop colors). Subsequently, the test piece leads to the second extraction through the oil soluble polymer coating, which causes the extraction (the second extraction) of the oil soluble heavy metal ion detecting agent-heavy metal ion complex to the test piece, thereby said test piece appearing to develop specific color. Furthermore, the test piece is taken out, and the color developed in said test piece is compared to the color piece on the Color Chart, thereby qualitatively and/or quantitatively identifying the heavy metal ion to be detected. Therefore, by combining the hydrophobic polymer coating and the heavy metal ion detecting agent on the substrate of said test piece, it is possible to increase the limit of detection of the heavy metal ions by double extraction (for example, heavy metal ions as low as 0.02 mg/L in the aqueous system can be detected). The reasons lie in that (1) compared to a simple aqueous solution system, the non-water soluble and oil soluble heavy metal ion detecting agent more readily reacts with heavy metal ions to form the heavy metal ion detecting agent-heavy metal chelate after being dissolved in an organic reagent and thoroughly mixed with the aqueous solution by means of agitation and the like; (2) compared to a simple aqueous solution system, since the volume of the organic solvent is far smaller than the volume of water in the aqueous system to be detected (such as a volume ratio of 1/20), the heavy metal ion in the aqueous solution can be enriched to some extent by the reaction between the heavy metal developer and the heavy metal ion (concentrated and enriched 20-fold under the conditions of complete reaction), thereby the organic solvent which accounts for only 1/20 volume can cause more significant color change of the test piece after being adsorbed by the test piece.

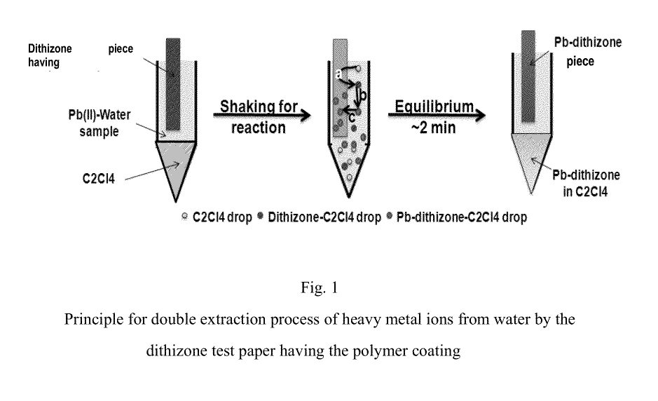

FIG. 1 illustrates the principle of the double extraction of the Pb(II) ion by the aforementioned test piece with the example that uses test paper as the substrate and dithizone as the heavy metal ion detecting agent. Firstly, the test piece is placed in a water/C₂Cl₄ mixture. Then the mixture is shaken to form a water sample/C₂Cl₄ emulsion. During the shaking, the emulsified C₂Cl₄ is adsorbed by the test piece due to the selective adsorption ability of the test piece, which causes a portion of the dithizone which initially adheres to the test piece to be dissolved in the droplet of the organic solvent (step a in FIG. 1), followed by the reaction between the green dithizone-C₂Cl₄ droplet and Pb(II) in water (step b in FIG. 1). Subsequently, the reacted red Pb-dithizone-C₂Cl₄ can be again adsorbed by the test piece (step c in FIG. 1). The whole process (step a to step c) will not stop until the completion of the Pb-dithizone reaction and the adsorption of the organic droplets by the test piece reaches a balance. Therefore, during the whole process, the Pb(II) in the water sample is first extracted to C₂Cl₄ and reacted with dithizone (the first extraction), followed by the further extraction of Pb-dithizone by the test piece (the second extraction). In such way, the limit of detection of the test piece can be greatly lowered by double extraction of Pb(II) from water, compared to that when a usual test paper is used, dithizone can only react with Pb(II) in the aqueous phase.

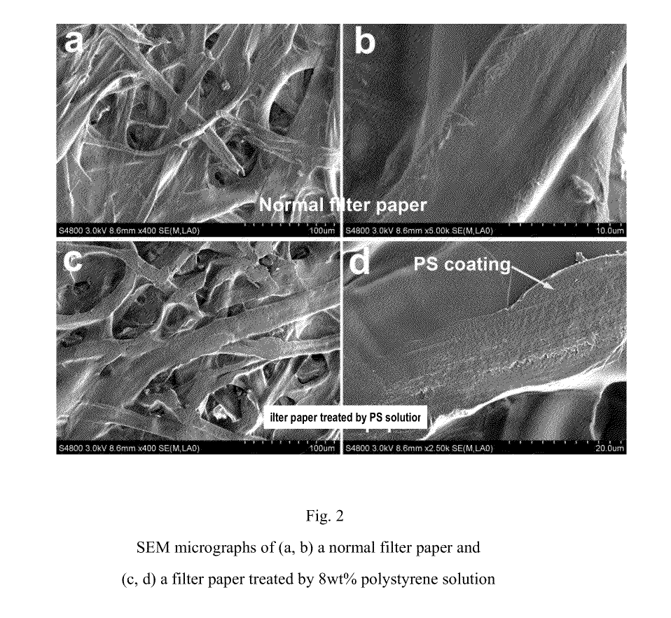

The double extraction process during color development in FIG. 1 occurs by the aforementioned structure of the test piece. The peculiarity of the structure of the test piece lies in that after the coating of the hydrophobic polymer on the substrate material, this coating of hydrophobic polymer covers the initial substrate and masks its property, which causes the whole test piece to be hydrophobic (lipophilic), thereby significantly changing the surface structure of the substrate material. FIG. 2 shows results of a scanning electronic microscopy (SEM). Based on the SEM results, compared to untreated normal paper (FIG. 2a, 2b), the microstructure of the normal paper (FIG. 2a,b) and filter paper coated with polymer (filter paper with a polymer coating) (FIG. 2c,d) can be clearly observed which shows that the treated filter paper (FIG. 2c, 2d) has a polymer coating on its surface fibers.

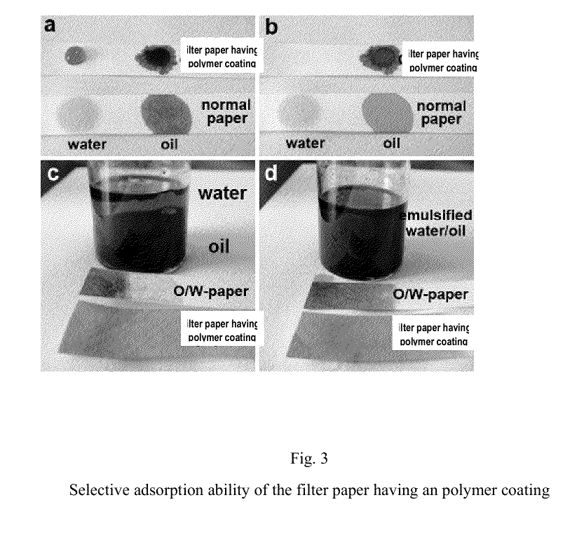

FIG. 3 shows the difference between the capability of the normal paper and the filter paper coated with a polymer to adsorb liquid with different surface energy, in particular the selective adsorption of the low surface free energy liquid (oils) in the high surface free energy liquid (water). In FIG. 3 (a, b), the left panel shows an aqueous solution, and the right panel shows a dithizone-organic solvent solution. According to FIG. 3a, the uncoated filter paper material has the ability of adsorbing the aqueous solution and the organic solvent solution; the filter paper coated with a polymer has lowered surface free energy and is only able to adsorb dithizone-organic solvent droplets; in contrast, the normal paper can adsorb both water and oil (FIG. 3a, b). These two types of filter paper are immersed in a water/organic solvent mixture, which further reveals that the treated filter paper has the ability of selective adsorption. No matter whether the mixture is layered or emulsified, the filter paper covered by the polymer coating (the filter paper with a polymer coating) only adsorb oil from water, but the normal paper is virtually covered by water (FIG. 3c,d). The FIGS. 3(a, b) are photos of the left side (red) water droplet and the right side (green) oil droplet on (a) the normal paper and the filter paper having a polystyrene coating (the test paper with a polymer coating), and (b) the filter paper having a polystyrene coating (the test paper with a polymer coating) which has the water droplet on it removed. FIGS. 3(c) and (d) are photos of the normal paper and the filter paper having a polystyrene coating after immersed into (c) a layered oil-water mixing system and (b) an emulsified oil-water mixing system and taken out.

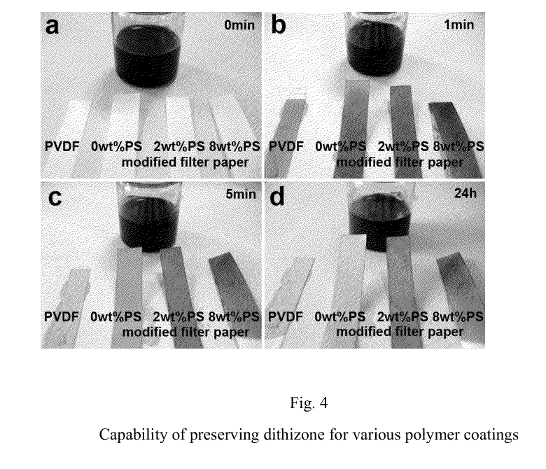

Among the test pieces with a polymer coating, it is preferred that the polymer layer is a polystyrene or poly(methyl methacrylate) layer. Test pieces coated with polystyrene or poly(methyl methacrylate) have higher capability to retain the heavy metal ion detecting agents. Take the heavy metal ion detecting agent dithizone as an example. FIG. 4 shows the capability of various dithizone test papers made from different filter paper or films to retain dithizone. Polyvinylidene difluoride (PVDF) film is a microporous filter having a hydrophobic surface for filtering organic solutions or suspensions, which can only adsorb organic solutions. Therefore, both PVDF film and normal paper (filter paper treated with 0 wt % styrene) are used as control. Filter paper is immersed into a dithizone-organic solvent solution (1 mg/mL) for 10 seconds, followed by drying in air and being placed in a place with good ventilation under natural light at room temperature for 24 hours to observe the color change. It can be seen that after 24 hours, the filter treated with 8 wt % polystyrene (PS) still remains green, indicating that the dithizone test paper formed from filter paper with PS retains dithizone better than a normal paper filter membrane.

The polymer coating in the test piece can be on all regions of the test piece, including both or either side of the test piece; or it can be on all or partial regions on one side of the test piece. The thickness of the polymer coating in the test piece is immaterial, as long as the polymer coating exists in the region to be detected.

Preferably, the level of the polymer in the polymer coating is usually immaterial. For example, when the polymer coating is obtained by dipping the test piece in the polymer solution, it only requires that the test piece is moistured thoroughly by the polymer solution.

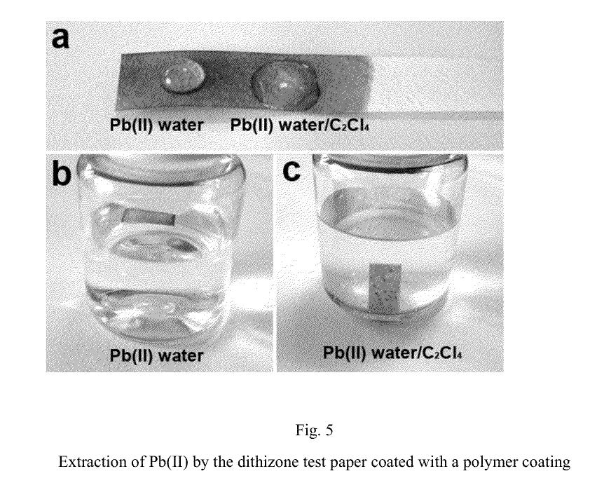

The test piece coated with polymer has complete water resistance, and is able to selectively adsorb liquid with low surface free energy (oil). FIG. 5 is a photo of the procedure of detecting Pb(II) in water with dithizone test paper with a polystyrene coating. Pb(NO₃)₂ is dissolved in deionized water to form a Pb(II) water sample (1×10⁻⁴ M). Due to the protective effect of the polystyrene layer, only the Pb(II) containing water dropped on the polystyrene coating of the dithizone test paper does not react with the dithizone on the test paper. However, after the double extraction of Pb(II) by the test paper, the droplet of Pb(II) water (water sample or aqueous system)/C₂Cl₄ mixture (volume ratio of water to C₂Cl₄=20/1) turns the dithizone test paper with a green polystyrene coating to red (FIG. 5a). The difference is further demonstrated by placing the dithizone test paper having polystyrene coating in different aqueous solutions and shaking. It can be seen that due to the protective effect of the polystyrene layer, the dithizone test paper with the polystyrene coating placed in Pb(II) water does not experience Pb(II)-dithizone reaction. However, the dithizone test paper with the polystyrene coating placed in the Pb(II) water sample/C₂Cl₄ mixture (the volume ratio of water to C₂Cl₄=20/1) experiences the reaction so that the green test paper completely turns to red. Moreover, it can be clearly seen that the droplets of reacted Pb-dithizone C₂Cl₄ is adsorbed by the test paper (FIG. 5c). This proves the principle of the detection by dithizone test paper with a polystyrene coating as illustrated in FIG. 1. FIG. 5 shows the extraction of Pb(II) by the dithizone test paper with a polystyrene coating. (a) Pb(II) water and Pb(II)-water C₂Cl₄ mixture droplets on the surface of the dithizone test paper with the polystyrene coating. Both droplets are dropped onto the test paper almost simultaneously and let stand for 2 minutes. (b) The dithizone test paper with the polystyrene coating on the surface of the Pb(II) water sample. The test paper is placed in a sample bottle which is shaken for 2 minutes. (c) The dithizone test paper with the polystyrene coating in the Pb(II) water sample/C₂Cl₄ mixture. The test paper is placed in a sample bottle which is shaken for 2 minutes. It can be clearly seen that the droplet of the Pb-dithizone C₂Cl₄ is adsorbed in the test paper.

According to one preferable embodiment of the invention, nanomaterials, for example, materials such as nano silica or nano titania, can be introduce into the heavy metal ion-detecting agent. Herein, the roughness and the specific surface area of the substrate can be increased so as to obtain oil/water separating materials with better hydrophobicity and lipophilicity, i.e., better ability of oil phase-selective adsorption, such as oil/water (or organic solvent/water) separating filter paper, after treatment.

Other materials, for example, adjuvants for improving the adhesion among the layers, can be added in the test piece as required. A color-developing adjuvant of the heavy metal ion detecting agent such as a color-developing adjuvant for the dithizone detecting agent can also be added. The adjuvants can be added in an amount as required.

During the preparation of the test piece, a polymer coating can be first applied to the substrate, for example, by coating the polymer coating, or by immersing the substrate in the polymer solution. The test piece with the formation of said polymer layer can be again coated with a layer of heavy metal ion detecting agent, such as a dithizone coating. Fore example, a normal paper is immersed in a polystyrene (PS)—toluene solution (8 wt %) for 30 seconds, and dried with an electronic dryer to form a filter paper covered with the polymer coating (the filter paper with a polymer filter).

According to one embodiment, the process for preparing the test piece comprises:

applying a layer of heavy metal ion detecting agent on the substrate; and coating a polymer protective layer on the heavy metal ion detecting agent layer. When an adsorbent material is added into the polymer coating, it can be introduced into the polymer coating before, concurrently with or after the application of the polymer coating.

Process for the Detection of the Heavy Metal Ions in an Aqueous System

Process for the detection of the heavy metal ions in an aqueous system according to the invention comprise bringing the test piece coated with the polymer coating and the heavy metal ion-detecting agent into contact with the aqueous system; shaking the aqueous system so as to contact with the test piece sufficiently; and observing whether the color of the test piece is changed. Preferably, the aqueous system to be detected contains an organic solvent. As required, organic solvent is additionally added into the aqueous system to be detected. Preferably, the organic solvent is selected from chlorine-containing C1-C6 alkane and chlorine-containing C1-C6 alkene, for example, CCl₄, CHCl₃ and C₂Cl₄.

In a particular embodiment, a process for detecting heavy metal ions in an aqueous system is provided, said process comprising:

(a) bringing the test piece for detecting the heavy metal ions in the aqueous system in contac with the aqueous system to be detected;

(b) shaking the aqueous system so as to contact sufficiently with the test piece; and

(c) observing whether the color of the test piece is changed.

When the color of the test piece changes, it is compared with the Color Chart to determine whether the heavy metal ion exists and/or the type of the heavy metal ion. When other interfering ions exist in the system, a masking agent can be added. The type of the masking agent can be determined by the interfering ion to be masked, such as Fe³⁺, Ca²⁺, or Mg²⁺.

According to a particular embodiment, a method for detecting a heavy metal ion is provided, including:

(a) adding an organic solvent into a solution to be detected;

(b) contacting the test piece with the solution to be detected;

(c) shaking the sample solution so as to fully contact with the test piece; and

(d) the color of the test piece changing.

Firstly, in step (a), the organic solvent added to the solution to be detected is an organic solvent that does not react with the heavy metal ion to be detected, preferably CCl₄, CHCl₃ or C₂Cl₄. Other organic solvents can also be used.

Subsequently, in step (b), the test piece coated with the polymer is placed into a water sample/organic solvent mixture, followed by shaking the mixture to form a water sample/organic solvent emulsion. During the shaking, a portion of the detecting agent that originally attaches to the test piece is dissolved into the droplet of the organic solvent to form an emulsified detecting agent. Due to the selectively adsorbing ability of the test piece, only the emulsified detecting agent is adsorbed by the test piece coated with the polymer, and not water droplet is adsorbed onto the test piece. Subsequently, the detecting agent for color development—organic solvent droplets react with the heavy metal ion to be detected, such as Pb(II) in water. In step (b), the reacted heavy metal ion detecting agent-heavy metal ion-organic solvent can again be adsorbed by the test piece. The whole process will not stop until the Pb-detecting agent reaction ends and the adsorption of the organic droplets by the test piece reaches a balance.

Therefore, during the whole process, the heavy metal ions in the water sample, such as Pb(II) is first extracted to the organic solvent to react with the detecting agent (the first extraction), and then the Pb-detecting agent is further extracted by the test piece coated with the polymer (the second extraction). In such a way, the limit of detection of the test piece coated with the polymer can be greatly lowered by double extraction of Pb(II) from water compared to that when normal paper is used, the detecting agent only reacts with Pb(II) in the aqueous phase.

In step (c), the detecting agent binding to the heavy metal ions is again extracted to the test piece and develops the specific color.

The color developed by the binding of the heavy metal ion detecting agent to the heavy metal ion is specific. A person of ordinary skill in the art can understand the difference caused by the type of the heavy metal ion detecting agent.

Because a polymer coating is combined in the test piece, the selective adsorbency, binding ability, and resistance to aqueous solvent during the detection of the heavy metal ion detecting agent is significantly elevated. Therefore, the duration of detection of the test piece of the invention is significantly shortened. Moreover, because of the protective effect of the polymer coating on the heavy metal detecting agent, the color of the heavy metal color developing agent in the test paper does not change within 24 hours of standing at room temperature in an open environment, indicating that no leakage or oxidation of the heavy metal color developing agent occurs.

The detection sensitivity of detecting the heavy metal ion is significantly increased due to the incorporation of the polymer coating layer in the polymer coating layer.

Kit for Detecting Heavy Metal Ions in an Aqueous System

A kit for detecting heavy metal ions in an aqueous system is provided, comprising a test piece coated with a polymer coating and a heavy metal ion detecting agent, an organic solvent able to dissolve the heavy metal ion detecting agent, and a Color Chart.

The organic solvent included in the kit is a solvent able to dissolve the heavy metal ion detecting agent and the heavy metal ion detecting agent-heavy metal ion complex, preferably selected from chlorine-containing C1-C6 alkane and chlorine-containing C2-C6 alkene, including CCl₄, CHCl₃ and C₂Cl₄. C₂Cl₄ is preferred in view of the environmental friendliness.

The Color Chart in the kit is not limited to one kind. Depending on the different type of the ions to be detected, the Color Chart may differ accordingly, which can be chosen by a person of ordinary skill in the art.

Moreover, the kit may further comprise an interfering ion masking agent, a color development adjuvant, or other adjuvants; and a means to extract the aqueous system sample to be detected.

Because the test piece of the invention has a hydrophobic polymer coating and has high sensitivity of detection to heavy metal ions, the kit may be made small, compact and portable, and can identify the heavy metal ions in the aqueous system to be detected lower than the usual limit of detection.

The Color Sensor for the Detection of Heavy Metal Ions in an Aqueous System

On the basis of the test piece coated with a polymer coating and a heavy metal ion detecting agent, a detection sensor that can be used to detect the heavy metal ions in an aqueous system is provided, which comprises a sample cell containing an organic solvent, a Color Chart, and a test piece color identification sensor.

The sensor for the detection of the heavy metal ions also comprises a sample preparation system, a set position for the test piece, a detection (color developing) segment for the test piece, an input/output system for the color developing data, a color sensor system to detect the developed color, a data processing/storage system and/or a data storage system.

In a particular embodiment, the ratio of the organic solvent to the aqueous system is such that the aqueous system is excessive in volume relative to the organic solvent.

In the detection by the sensor for detecting the heavy metal ions, the reader reads R value (red), G value (green), and B value (blue). The type and/or concentration of the heavy metal ion are determined according to the reading of the test piece color identification sensor.

Specifically, according to the principle of color sensor, the test piece color identification sensor will use a color sensor chip, such as TCS 230 chip produced by TAOS (Texas Advanced Optoelectronic Solutions) to identify the heavy metal ion based on the color sensing principle according to the RGB data of specific colors. This color sensor has a simple structure, which projects white light LED toward the object; the color sensor chip receives the reflected light, and converts the light signal into digital RGB data signal using red/green/blue filter and the CMOS circuit embedded in the chip. Moreover, the type and concentration of the heavy metal ions can be determined by a combination of a digital camera and a smart phone that has photo-taking function with a graphic RGB analyzing software based on PC or smart phone operating system. The graphic system takes pictures of objects, and the RGB analyzing software analyzes the photo to obtain the RGB data of the objects. Because the color sensor is highly integrated and modulated, which can read almost every kind of color, a portable test paper detector produced based on the color sensor system is more convenient and simpler than a traditional test paper reflectometer.

The color sensor of the invention is a portable test paper detector by combining the color sensor system with the colorimetric test paper for the rapid in situ detection of the heavy metal ions or residual pesticides in water source, soil, or vegetables and fruits.

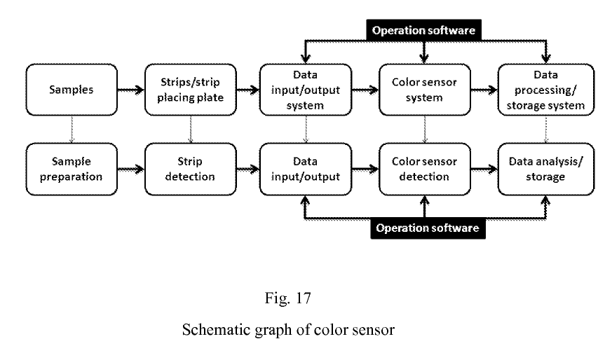

FIG. 17 is the structure of a portable test paper detector based on the color sensor system, and a diagram of the detection procedure using said detector. Said portable test paper detector comprises five parts: multiple test paper/test paper carrier slip, a data input/output system component, a color sensor system component, a data processing/storage system component, and an operating software. Accordingly, the detection process comprises five steps: 1. preparing the sample; 2. detecting the sample with the test paper, and putting the reacted test paper on the test paper carrier slip; 3. selecting the specific detection program in the operating system according to the type of the test paper (for example, the dithizone test paper for the detection of heavy metal ions or AChE enzyme test paper for the detection of residual pesticides); 4. reading the color of the test paper by the color sensor; and 5. comparing the test paper RGB data and the stored reference RGB data by the data processing/storage system, and storing and finally outputting the detection results.

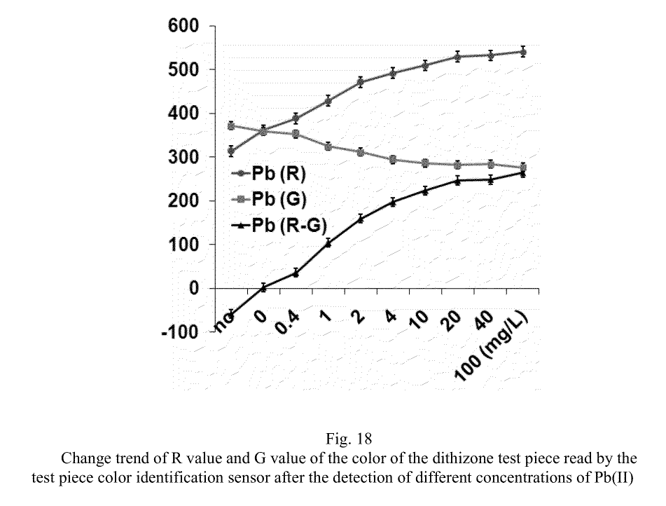

FIG. 18 is a trend graph for the change of R value and G value of the color of the dithizone test piece after the detection of different concentration of Pb(II) by the test piece color identification sensor. From the figure, it can be clearly seen that the R values and G values of the color of the test piece corresponds one by one to the different concentrations of Pb(II). The corresponding R values and G values of the test paper in these fixed concentrations can be saved as reference values.

On the other hand, the instrument of the present application can be assembled to a detection assembly comprising the instrument of the present application and a container filled with the coloring agent solution. After the detection region of the detection material of said instrument is contacted with the aqueous solution to be detected, it can be inserted into the coloring agent solution for in situ determination so as to facilitate the detection process. The coloring agent can be a water soluble coloring agent, including a water soluble dye such as red ink, blue ink, and the like.

In another specific embodiment, this detection assembly can further include a container filled with a water-soluble solubilizer, and/or an electrolysis equipment. As mentioned above, duration of detection can be shortened and the sensitivity of detection can be elevated by using a water soluble solubilizer and/or electrified procedure. Said water soluble solubilizer includes water soluble alcohols, such as C1-C4 aliphatic alcohols, preferably ethanol. By using this electrolysis equipment, the electrified procedure can be conducted during detection. This electrolysis equipment can comprise an electrolyte tank, a reference electrode, a counter electrode, and a power supply.

The detection assembly can be used for the following detection: taking a sample of the aqueous solution to be detected and poured it into the electrolyte tank of the electrolysis equipment, optionally adding a certain amount of water soluble solubilizer; immersing the detection region of the detection material into said aqueous solution to be detected; then connecting the electrolysis equipment to apply a negative voltage to the detection material for the electrified procedure; subsequently, taking out the detection material, and immersing the detection region of the detection material after contacting with the aqueous solution to be detected into a coloring agent solution; and determining whether the heavy metal ion exists in the aqueous solution to be detected by whether said detection region adsorbs said coloring agent solution (that is, whether color change appears).

As mentioned above, the instrument and the detection assembly of the present application can in situ detect the heavy metal ions in water with low cost. The heavy metal ions primarily include ions of cadmium (Cd), lead (Pb), mercury (Hg), copper (Cu), and zinc (Zn); especially the common ions of cadmium (Cd), lead (Pb), mercury (Hg) and the like that are highly hazardous to human body. Moreover, the instrument and the detection assembly of the present application can be conveniently used without constraints posed by environmental conditions.

These specific illustrations are merely intended to provide a person skilled in the art with aid or teaching that may be needed for carrying out the invention, and is not intended to limit the invention by any means. Hereinafter, the invention is described in more details by way of examples. It should however be understood that these examples are for illustration only and are in no way limiting. Unless described otherwise, all raw materials used are commercially available.

### EXAMPLES

### Example 1

Detection of Pb(II) in water using the normal dithizone test papers made by the normal filter paper substrate and using the dithizone test pieces (test papers) made by the filter paper substrate coated with a polymer coating.

The filter paper coated with polystyrene (PS) coating (filter paper having a polymer coating) was prepared by dipping the filter paper into 8 wt % polystyrene (PS) solution in toluene for 30 s and then drying by a hair dryer. The normal dithizone test paper or the dithizone test paper having the polymer coating was prepared, respectively, by dipping the normal filter paper or the filter paper having the polymer coating into 1 mg/mL dithizone-C₂Cl₄ solution for 10 s and then drying at room temperature.

The detection of Pb(II) ions was performed by placing the normal dithizone test paper or the dithizone test paper having the polymer coating into disposable sample bottles which contain 10 mL of aqueous Pb(II) solution with various concentrations (prepared from Pb(NO₃)₂ and deionized water) or 10 mL of aqueous Pb(II) solution/C₂Cl₄ mixture (volume ratio of water to C₂Cl₄=20/1). After the sample bottles were shaken for 2 min, the test papers were taken out to observe the color change.

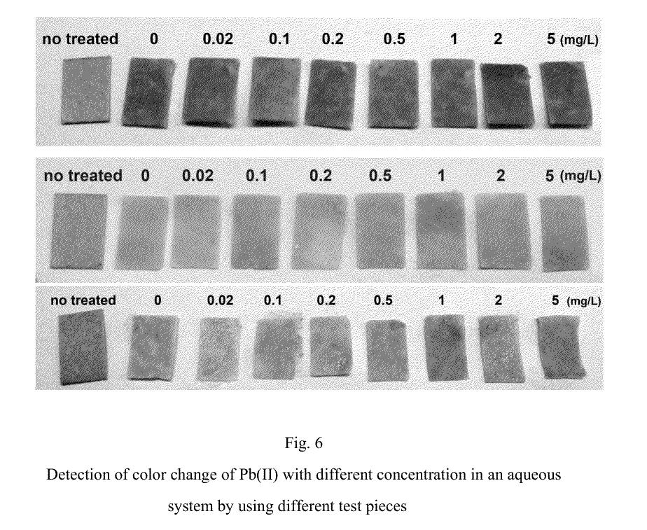

FIG. 6 depicts the color change of the normal dithizone test paper or the dithizone test paper having the polymer coating after being treated with an aqueous Pb(II) solution or an aqueous Pb(II) solution/C₂Cl₄ mixture. FIG. 6 shows the detection of Pb(II) in an aqueous solution using the normal dithizone test pieces (test papers) made by the normal filter paper and using the dithizone test papers made by the filter paper substrate having the polymer coating. (Upper) The photos show the color change of the test papers obtained after the reaction by placing the normal dithizone test papers into the aqueous Pb(II) solution with various concentrations. (Middle) The photos show the color change of the test papers obtained after the reaction by placing the normal dithizone test papers into the aqueous Pb(II) solution/C₂Cl₄ mixture with various concentrations. (Lower) The photos show the color change of the test papers obtained after the reaction by placing the dithizone test papers made by the filter paper having the polymer coating into the aqueous Pb(II) solution/C₂Cl₄ mixture with various concentrations.

It is apparent that the dithizone test pieces placed into the aqueous Pb(II) solution/C₂Cl₄ mixture have the most distinguished color change. It was found that the addition of C₂Cl₄ into water can improve slightly the detection effect of Pb(II) for the normal dithizone test piece, but still much worse than that for the dithizone pieces having the polymer coating. When the dithizone test paper having the polymer coating prepared by 8 wt % PS and 1 mg/mL dithizone-C₂Cl₄ aqueous solution is used to detect Pb(II), the lower detection limit thereof is 0.02 mg/L, and the detection range for Pb(II) is 0.02-5 mg/L.

### Example 2

Detection of Cd(II) in water using the dithizone test paper having the polymer coating.

The filter paper having the polymer coating was prepared by dipping the filter paper into 8 wt % polystyrene (PS)-toluene solution for 30 s and then drying by a hair dryer. The dithizone test piece having the polymer coating was prepared by dipping the filter paper having the polymer coating into 1 mg/mL dithizone-CCl₄ solution for 10 s and then drying at room temperature.

The detection of Cd(II) ions was performed by placing the dithizone test paper having the polymer coating into disposable bottles which contain 10 mL aqueous Cd(II) solution (prepared from CdCl₂ and deionized water)/C₂Cl₄ mixture (volume ratio of water to C₂Cl₄=20/1). After the bottles were shaken for 2 min, the test papers were taken out to observe the color change.

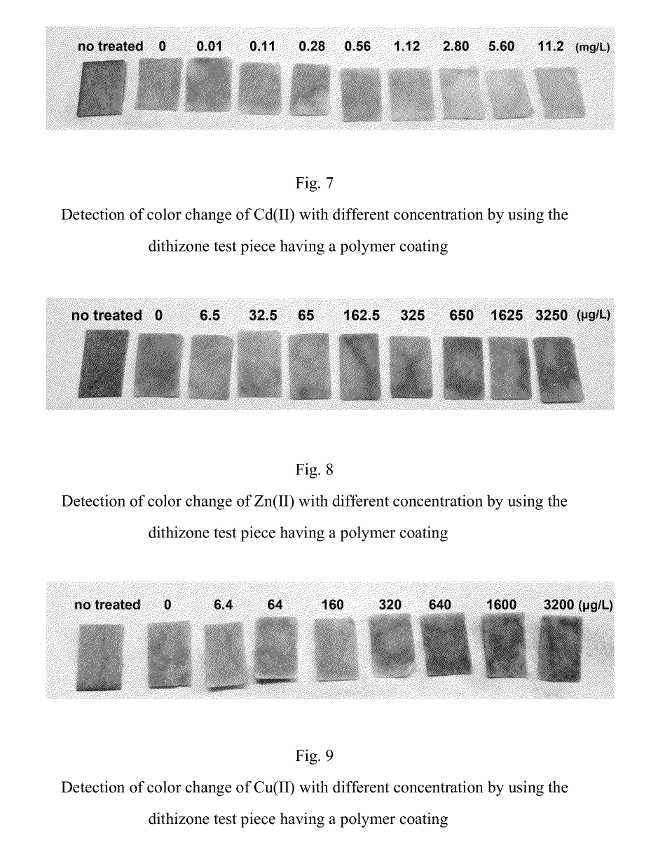

FIG. 7 depicts the color change of the dithizone piece having the polymer coating after being treated with an aqueous Cd(II) solution/C₂Cl₄ mixture. When the dithizone piece having the polymer coating prepared by 8 wt % PS and 1 mg/mL dithizone-CCl₄ solution is used to detect Cd(II), the lower detection limit thereof is 0.01 mg/L, and the detection range for Cd(II) is 0.01˜11.2 mg/L.

### Example 3

Detection of Zn(II) in water using the dithizone test paper having the polymer coating.

The filter paper having the polymer coating was prepared by dipping the filter paper into 8 wt % polystyrene (PS)-toluene solution for 30 s and then drying by a hair dryer. The dithizone test paper having the polymer coating was prepared by dipping the filter paper having the polymer coating into 1 mg/mL dithizone-CCl₄ solution for 10 s, and then drying at room temperature.

The detection of Zn(II) ions was performed by placing the dithizone test paper having the polymer coating into disposable bottles which contain 10 mL aqueous Zn(II) solution (prepared from ZnSO₄ and deionized water)/CCl₄ mixture (volume ratio of water to CCl₄=20/1). After the bottles were shaken for 2 min, the test papers were taken out to observe the color change.

FIG. 8 depicts the color change of the dithizone test paper having the polymer coating after being treated with an aqueous Zn(II) solution/CCl₄ mixture. When the dithizone piece having the polymer coating prepared by 8 wt % PS and 1 mg/mL dithizone-CCl₄ solution is used to detect Zn(II), the lower detection limit thereof is 6.5 μg/L, and the Zn(II) detection range is 6.5˜3250 μg/L.

### Example 4

Detection of Cu(II) in water using the dithizone test paper having the polymer coating.

The filter paper having the polymer coating was prepared by dipping the filter paper into 8 wt % polystyrene (PS)-toluene solution for 30 s and then drying by a hair dryer. The dithizone test paper having the polymer coating was prepared by dipping the filter paper having the polymer coating into 1 mg/mL dithizone-C₂Cl₄ solution for 10 s and then drying at room temperature.

The detection of Cu(II) ions was performed by placing the dithizone test paper having the polymer coating into disposable bottles which contain 10 mL aqueous Cu(II) solution (prepared from Cu(NO₃)₂ and deionized water)/C₂Cl₄ mixture (volume ratio of water to C₂Cl₄=20/1). After the bottles were shaken for 2 min, the test papers were taken out to observe the color change.

FIG. 9 depicts the color change of the dithizone test paper having the polymer coating after being treated with aqueous Cu(II) solution/C₂Cl₄ mixture. When the dithizone test paper having the polymer coating prepared by 8 wt % PS and 1 mg/mL dithizone-C₂Cl₄ solution is used to detect Cu(II). The lower detection limit thereof is 6.4 μg/L, and the detection range for Cu(II) is 6.4˜3200 μg/L.

### Example 5

Detection of Hg(II) in water using the dithizone test paper having the polymer coating.

The filter paper having the polymer coating was prepared by dipping the filter paper into 8 wt % polystyrene (PS)-toluene solution for 30 s and then drying by a hair dryer. The dithizone test paper having the polymer coating was prepared by dipping the filter paper having the polymer coating into 1 mg/ mL dithizone-C₂Cl₄ solution for 10 s and then drying at room temperature.

The detection of Hg(II) ions was performed by placing the dithizone test paper having the polymer coating into disposable bottles which contain 10 mL Hg(II) aqueous solution (prepared from HgSO₄ and deionized water with pH value adjusted to 1˜2 by HCl)/C₂Cl₄ mixture (volume ratio of water to C₂Cl₄=20/1). After the bottles were shaken for 2 min, the test papers were taken out and to observe the color change.

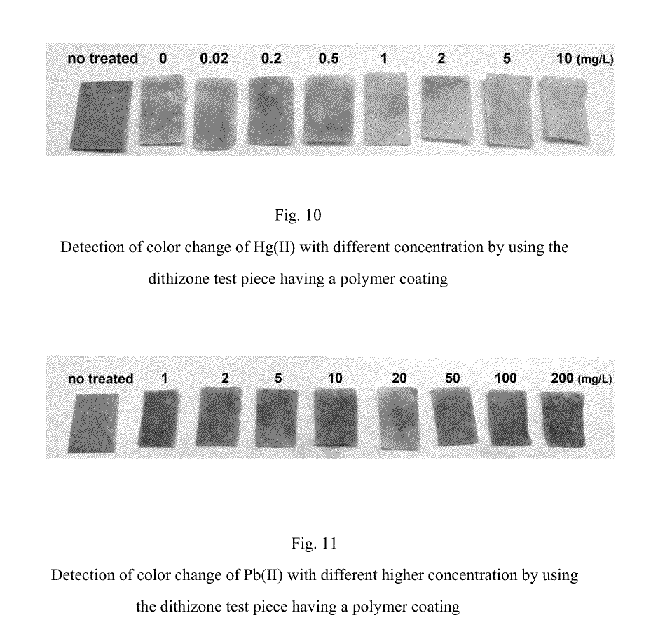

FIG. 10 depicts the color change of the dithizone test paper having the polymer coating after being treated with aqueous Hg(II) solution/C₂Cl₄ mixture. When the dithizone test paper having the polymer coating prepared by 8 wt % PS and 1 mg/mL dithizone-C₂Cl₄ solution is used to detect Hg(II), the lower detection limit thereof is 0.02 mg/L, and the detection range for Hg(II) is 0.02˜10 mg/L.

### Example 6

Detection of Pb(II) with higher concentrations in water using the dithizone test paper having the polymer coating.

The filter paper having the polymer coating was prepared by dipping the filter paper into 8 wt % polystyrene (PS)-toluene solution for 30 s and then drying by a hair dryer. The dithizone test paper having the polymer coating was prepared by dipping the filter paper having the polymer coating into 2 mg/mL dithizone-C₂Cl₄ solution for 10 s and then drying at room temperature.

The detection of Pb(II) ions was performed by placing the dithizone test paper having the polymer coating into disposable bottles which contain 10 mL aqueous Pb(II) solution (prepared from Pb(NO₃)₂ and deionized water)/C₂Cl₄ mixture (volume ratio of water to C₂Cl₄=20/1). After the bottles were shaken for 1 min, the test papers were taken out and to observe the color change.

FIG. 11 depicts the color change of the dithizone test paper having the polymer coating after being treated with aqueous Pb(II) solution/C₂Cl₄ mixture. The detection range changed significantly due to the increase of the loaded dithizone in the dithizone test paper having the polymer coating. The detection range for Pb(II) by using the dithizone test paper prepared by 8 wt % PS and 2 mg/mL dithizone-C₂Cl₄ solution is 1˜200 mg/L.

### Example 7

Alternative polymer materials for preparation of the dithizone test paper having the polymer coating.

The filter paper having the polymer coating was prepared by dipping the filter paper into 8 wt % poly(methylmethacrylate) (PMMA)-tetrahydrofuran solution for 30 s and then drying by a hair dryer. The dithizone test paper having the polymer coating was prepared by dipping the filter paper having the polymer coating into 1 mg/mL dithizone-C₂Cl₄ solution for lOs and then drying at room temperature.

The red aqueous solution is prepared by diluting a red ink in deionized water. The green organic solution is 1 mg/mL of dithizone-C₂Cl₄ solution. The aqueous Zn(II) solution is prepared from ZnSO₄ and deionized water.

The selective absorption test was conducted by dropping the red water and the green oil onto the normal filter paper and the PMMA coated filter paper having the polymer coating (paper having the polymer coating), standing for 2 min, and then removing the water drops from the paper having the polymer coating.

The heavy metal detection capability test was conducted by dropping deionized water/C₂Cl₄ mixture (20/1) and Zn(II) water/C₂Cl₄ mixture (20/1) on the PMMA coated dithizone test paper having the polymer coating, and then standing for 2 min.

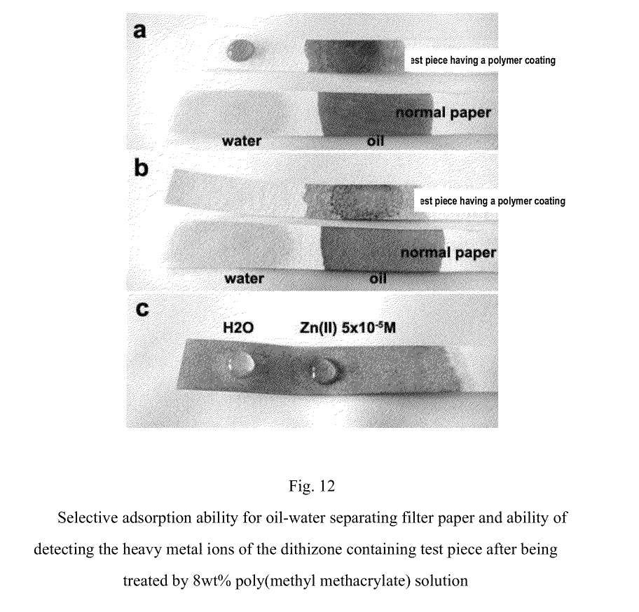

FIG. 12 depicts the selective absorption ability of the PMMA coated separation filter paper and the capability of detecting heavy metal ions for the PMMA coated separation filter paper having the polymer coating. FIGS. 12a and 12b show that the filter paper having the polymer coating prepared by the PMMA solution also possesses the selective absorption ability towards water and C₂Cl₄. FIG. 12c depicts that the PMMA coated dithizone test paper having the polymer coating has the capability of the detection of heavy metal ions as well.

### Example 8

Multi-sample detection using the dithizone paper having the polymer coating.

The filter paper having the polymer coating was prepared by dipping the filter paper into 8 wt % polystyrene (PS)-toluene solution for 30 s and then drying by a hair dryer. The dithizone paper having the polymer coating was prepared by dipping the filter paper having the polymer coating into 1 mg/mL dithizone-C₂Cl₄ solution for 10 s and then drying at room temperature.

The aqueous Cd(II) solution is prepared by dissolving the CdCl₂ in deionized water. The aqueous Pb(II) solution is prepared by dissolving the Pb(NO₃)₂ in deionized water. The aqueous Zn(II) solution is prepared by dissolving the ZnSO₄ in deionized water. The aqueous Cu(II) solution is prepared by dissolving the Cu(NO₃)₂ in deionized water.

The multi-sample detection was conducted by dropping 50 μL heavy metal ion solution in water/C₂Cl₄ mixture (20/1) on the same dithizone test paper having the polymer coating and standing for 5 min.

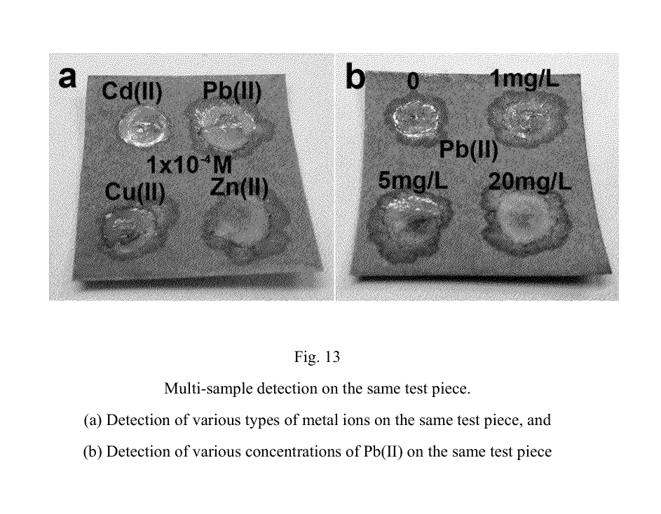

FIG. 13 depicts the photos of the detection of different types of heavy metal ions at the same molar concentration and aqueous Pb(II) solutions with different concentrations using the dithizone paper having the polymer coating. In FIG. 13, the multi-sample detection was carried out in the different aqueous solutions on the same the dithizone test paper having the polymer coating: (a) different types of heavy metal ions and (b) aqueous Pb(II) solutions with different concentrations. Different colors correspond to different types of heavy metal ions (FIG. 13a), and different shade degrees of red color correspond to aqueous Pb(II) solutions with different concentrations (FIG. 13b).

### Example 9

Alternative paper substrates for the preparation of the dithizone test paper.

The paper having the polymer coating was prepared by dipping the normal A4 printing paper into 8 wt % polystyrene (PS)-toluene solution for 30 s and then drying by a hair dryer. The dithizone test paper having the polymer coating was prepared by dipping the normal A4 paper having the polymer coating into 1 mg/mL dithizone-C₂Cl₄ solution for 10 s and then drying at room temperature.

The red aqueous solution (water) was prepared by diluting a red ink in deionized water. The green organic solution was 1 mg/mL dithizone-CCl₄ solution.

The aqueous Cd(II) solution was prepared by dissolving CdCl₂ in deionized water. The aqueous Pb(II) solution was prepared by dissolving the Pb(NO₃)₂ in deionized water. The aqueous Zn(II) solution was prepared by dissolving the ZnSO₄ in deionized water. The aqueous Cu(II) solution was prepared by dissolving Cu(NO₃)₂ in deionized water.

The selective absorption test was conducted by dropping the red water and the green oil onto the normal A4 paper (normal paper) and the A4 paper having the polymer coating (the paper having the polymer coating), and standing for 2 min, then removing the water drops from the paper having the polymer coating.

The multi-sample detection was conducted by dropping heavy metal ion solution in water/C₂Cl₄ mixture (20/1) on the dithizone test paper having the polymer coating prepared by A4 printing paper, and then standing for 5 min.

FIGS. 14a and 14b depict the selective absorption ability towards water and CCl₄ for the A4 paper having the polymer coating. FIG. 14c shows that the dithizone test paper having the polymer coating obtained by treating the normal A4 printing paper with 8 wt % PS solution and 1 mg/mL dithizone-C₂Cl₄ solution has the ability of multi-sample detection.

### Example 10

Detection of Ag(I) using the rose red silver test paper having the polymer coating.

The paper having the polymer coating was prepared by dipping the normal A4 printing paper into 8 wt % polystyrene (PS)-toluene solution for 30 s and then drying by a hair dryer. The normal p-dimethylaminobenzal rhodanine test paper and the p-dimethylaminobenzal rhodanine test paper having the polymer coating was prepared respectively by dipping the normal A4 paper or the A4 paper having the polymer coating into 0.5 mg/mL p-dimethylaminobenzal rhodanine solution in acetone/C₂Cl₄ (the volume ratio is 1/1) for 10 s, and then drying at room temperature. The aqueous Ag(I) ion solution was prepared by dissolving AgNO₃ indeionized water.

The detection of Ag(I) was conducted by dropping the deionized water/C₂Cl₄ mixture (20/1) and the aqueous Ag(I) solution/C₂Cl₄ mixture (20/1) on the p-dimethylaminobenzal rhodanine test paper having the polymer coating or the normal p-dimethylaminobenzal rhodanine test paper, and then standing for 5 min.

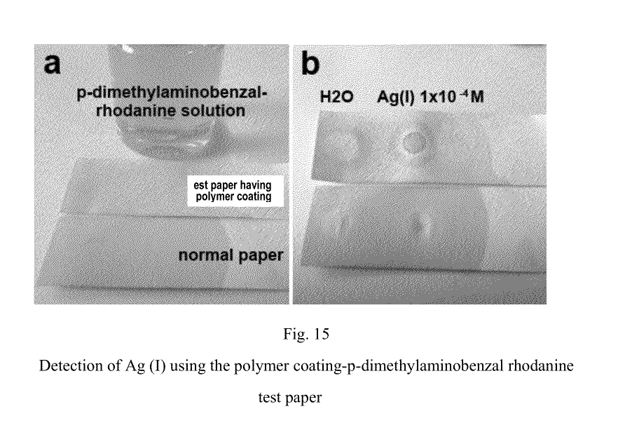

FIG. 15a depicts the p-dimethylaminobenzal rhodanine preserving capability of the normal A4 paper (normal paper) and the A4 paper having the polymer coating (the paper having the polymer coating). In FIG. 15, the p-dimethylaminobenzal rhodanine test paper having the polymer coating was employed in the detection of Ag(I). (a) The test paper for the detection of Ag(I) was prepared from the p-dimethylaminobenzal rhodanine solution in acetone/C₂Cl₄ and the PS-coated A4 paper or the normal A4 paper. (b) the p-dimethylaminobenzal rhodanine test paper having the polymer coating was employed in the detection of Ag(I).

It is obvious from the colors of the prepared test papers and the p-dimethylaminobenzal rhodanine solution that the paper having the polymer paper has a better heavy metal reagent preserving capability than the normal paper. FIG. 15b depicts the detection of Ag(I) using these two types of test papers. It is clear that the p-dimethylaminobenzal rhodanine test paper having the polymer coating has a better color contrast than the normal test paper.

### Example 11

The stability of the dithizone test paper having the polymer coating.

The filter paper having the polymer coating was prepared by dipping the filter paper into 8 wt % polystyrene (PS)-toluene solution for 30 s and then drying by a hair dryer. The normal dithizone test paper and the dithizone test paper having the polymer coating were prepared, respectively, by dipping the normal filter paper or the filter paper having the polymer coating into 1 mg/mL dithizone-C₂Cl₄ solution for 10 s and then drying at room temperature.

The stability test was conducted by comparing the colors of four dithizone test papers having the polymer coating after different treatments. One test paper was prepared by dipping the filter paper having the polymer coating into the newly prepared dithizone-C₂Cl₄ solution (newly prepared one). Another test paper was prepared by dipping the filter paper having the polymer coating into the newly prepared dithizone-C₂Cl₄ solution and placing in the open air at room temperature for a month (the test piece after placing in the open air for 1 month). The third one was prepared by dipping the filter paper having the polymer coating into the newly prepared dithizone-C₂Cl₄ solution and placing in a sealed brown bottle shielded from the light at room temperature for a month (the test piece shielded from the light after 1 month). And the last one was prepared by dipping the filter paper having the polymer coating into the dithizone-C₂Cl₄ solution which had been kept in a sealed brown bottle shielded from the light at room temperature for a month (the dithizone solution after shielding from the light for 1 month).

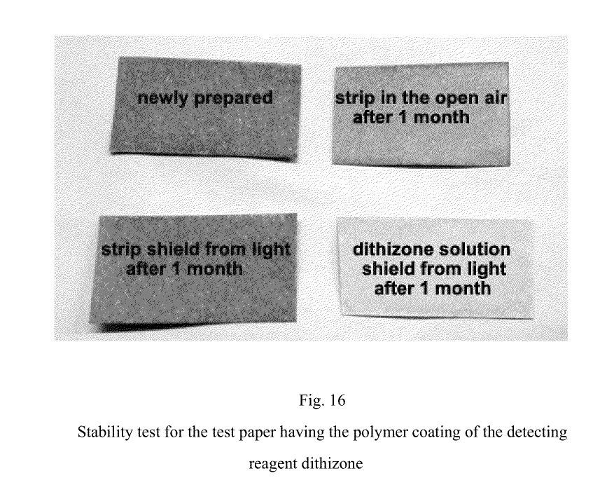

FIG. 16 depicts the stability of the dithizone test paper having the polymer coating. From the color, it is apparent that, under the protection of the polymer layer and shielding from the light, dithizone can be preserved in the filter paper having the polymer coating very well. And even in the open air, the dithizone can be also preserved better than the dithizone solution which is shielded from the light.

### Example 12

Change trend of R value (Red Value) and G value (Green Value) of the color of the dithizone test paper having the polymer coating read by the test piece color identification sensor after the detection of Pb(II) at different concentrations.

The filter paper having the polymer coating was prepared by dipping the filter paper into 8 wt % polystyrene (PS)-toluene solution for 30 s and then drying by a hair dryer. The dithizone test paper having the polymer coating was prepared by dipping the filter paper having the polymer coating into 2 mg/mL dithizone-C₂Cl₄ solution for 10 s, and then drying at room temperature. The aqueous Pb(II) solution was prepared by dissolving Pb(NO₃)₂ in deionized water.

The detection of Pb(II) ions was performed by placing the dithizone test paper having the polymer coating into disposable sample bottles which contain 10 mL of aqueous Pb(II) solution/C₂Cl₄ mixture (volume ratio of water to C₂Cl₄=20/1). After the sample bottles were shaken for 1 min, the test papers were taken out for reading the color values by use of the color identification sensor.

FIG. 18 depicts the trend of the color change of test paper after the detection of Pb(II), taking the detection of Pb(II) by use of the dithizone test paper having the polymer coating as an example. The color of the dithizone test paper having the polymer coating changes from the green color of dithizone into the red color of Pb-dithizone, and the shade of the red color are continuously deepened with the increase of the Pb(II) concentration, as shown by the trend graph of the color: R value has a trend of increasing with the increase of the Pb(II) concentration; G value has a trend of decreasing with the increase of the Pb(II) concentration, and the difference between the R value and B value tends to increase. Therefore, the R value and G value corresponding to a certain Pb(II) concentration can be set as the reference value for the determination of the Pb(II) concentration (range) in the real water sample by comparing with the color values of the test paper after the detection of Pb(II) in a practical detection.

The embodiment of the invention being thus described, it will be obvious that the same may be varied in many ways. Such variations are not to be regarded as a departure from the spirit and scope of the invention, and all such modifications as would be obvious to those skilled in the art are intended to be included within the scope of the following claims.

1. A test piece for detecting heavy metal ions, comprising a substrate, a polymer coating layer and a layer of heavy metal ion-detecting agent, wherein the polymer coating layer is provided such that the surface of the test piece is hydrophobic.

2. The test piece according to claim 1, wherein the polymer in the polymer coating layer is selected from the group consisting of polyethylene; polyvinyl chloride; polystyrene; polypropylene; polybutene; polyisobutylene; polyformaldehyde; polyamides; polycarbonates; polylactic acid; polytetrafluoroethylene; poly(ethylene terephthalate); epoxy resins; phenolic resins; polyurethanes; polyacrylonitrile-butadiene-styrene; and poly(methyl methacrylate).

3. The test piece according to claim 1, wherein the polymer coating layer is disposed on the substrate, and the detecting agent in the layer of the heavy metal ion-detecting agent is distributed within the polymer coating layer.

4. The test piece according to claim 1, wherein the heavy metal ion-detecting agent in the layer of the heavy metal ion-detecting agent is an oil-soluble detecting agent for heavy metal ions.

5. The test piece according to claim 1, wherein the heavy metal ion-detecting agent is selected from the group consisting of dithizone, p-dimethylaminobenzal rhodanine (rose red silver reagent), diphenylcarbohydrazide, or a developer based on triphenylmethane.

6. The test piece according to claim 1, wherein the test piece is used for the detection of the heavy metal ions in an aqueous system, and the aqueous system contains an organic solvent capable of dissolving the oil soluble heavy metal ion-detecting agent, or an organic solvent capable of dissolving the oil soluble heavy metal ion-detecting agent is added to the aqueous system.

7. The test piece according to claim 6, wherein the organic solvent is selected from the group consisting of chlorine-containing alkane or chlorine-containing alkene, preferably at least one of CCl₄, CHCl₃ and C₂Cl₄.

8. A process for detecting heavy metal ions in an aqueous system, comprising:
a. ging the test piece according to claim 1 into contact with the aqueous system to be detected;
b. shaking the aqueous system so as to contact with the test piece sufficiently; and
c. observing whether the color of the test piece is changed.

9. The process according to claim 8, comprising:
comparing the colored test piece with a color chart to determine whether a heavy metal ion is present and/or determine the species of heavy metal ion.

10. The process according to claim 9, wherein the aqueous system to be detected contains an organic solvent capable of dissolving the oil soluble heavy metal ion-detecting agent, or an organic solvent capable of dissolving the oil soluble heavy metal ion-detecting agent is added to the aqueous system to be detected.

11. The process according to claim 9, wherein the organic solvent is selected from the group consisting of chlorine-containing alkane or chlorine-containing alkene, preferably from at least one of CCl₄, CHCl₃ and C₂Cl₄.

12. A kit for detecting heavy metal ions in an aqueous system, comprising the test piece according to claim 1 and a color chart.

13. The kit according to claim 12, comprising an organic solvent capable of dissolving the oil soluble heavy metal ion-detecting agent.

14. The kit according to claim 13, wherein the organic solvent is selected from chlorine-containing alkane or chlorine-containing alkene, preferably from at least one of CCl₄, CHCl₃ and C₂Cl₄.

15. The kit according to claim 13, wherein a volume ratio of the organic solvent to the aqueous system is between 1:10 and 1:50.

16. A detecting sensor for detecting heavy metal ions in an aqueous system, comprising the test piece according to claim 1.

17. The detecting sensor according to claim 16, comprising a test piece color identification sensor, a color chart, and optionally a sample cell containing an organic solvent.

18. The heavy metal ion-detecting sensor according to claim 17, wherein the ratio of the organic solvent to the aqueous system is such that the aqueous system is excessive in volume relative to the organic solvent, and preferably a ratio in volume of the organic solvent to the aqueous system is between 1:10 and 1:50.

19. The heavy metal ion-detecting sensor according to claim 17, wherein the test piece color identification sensor is capable of identifying the concentration of the respective heavy metal ions by comparing the color values read by itself, such as R value (red), G value (green) and B value (blue), with the reference color values.

20. The heavy metal ion-detecting sensor according to claim 17, wherein the species of the heavy metal ions and/or the concentration of the heavy metal ions are determined according to the reading on the color recognition sensor of the test piece.
